(19) United States
(12) Patent Application Publication
(10) Pub. No.: US 2006/0219627 A1
(43) Pub. Date: Oct. 5, 2006

(54) MEMS filter module with concentric filtering walls

(76) Inventors: M. Steven Rodgers, Albuquerque, NM (US); Norman F. Smith, Albuquerque, NM (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(21) Appl. No.: 11/095,995

(22) Filed: Mar. 31, 2005

(51) Int. Cl. B01D 29/07; B01D 63/00; A61F 2/16

(52) U.S. Cl. 210/498; 210/321.84; 623/6.11; 604/891.1

(57) ABSTRACT

A bidirectional flow MEMS filter module (40) is disclosed that uses a plurality of concentrically disposed filtering walls (60). The MEMS filter module (40) includes a first plate (44) having a plurality of first flow ports (48), as well as a second plate (52) having a plurality of second flow ports (56) that is spaced from and interconnected with the first plate (44). The above-noted filtering walls (60) extend from the second plate (52) at least toward the first plate (44). The first plate (44) and each filtering wall (60) are spaced from each other to define a filter trap (64). All flow through the MEMS filter module (40) must pass through at least one filter trap (64) prior to exiting the MEMS filter module (40), either through one or more of the first flow ports (48) or through one or more of the second flow ports (56).

### FIELD OF THE INVENTION

The present invention generally relates to the field of microfabricated filters and, more particularly, to microfabricated filters that accommodate a desirably high flow rate and that may be utilized in a glaucoma implant or the like.

### BACKGROUND OF THE INVENTION

High internal pressure within the eye can damage the optic nerve and lead to blindness. There are two primary chambers in the eye—an anterior chamber and a vitreous body that are generally separated by a lens. Aqueous humor exists within the anterior chamber, while vitreous humor exists in the vitreous body. Generally, an increase in the internal pressure within the eye is caused by more fluid being generated within the eye than is being discharged by the eye. The general consensus is that it is excess fluid within the anterior chamber of the eye that is the main contributor to an elevated intraocular pressure.

One proposed solution to addressing high internal pressure within the eye is to install an implant. Implants are typically directed through a wall of the patient's eye so as to fluidly connect the anterior chamber with an exterior location on the eye. There are a number of issues with implants of this type. One is the ability of the implant to respond to changes in the internal pressure within the eye in a manner that reduces the potential for damaging the optic nerve. Another is the ability of the implant to reduce the potential for bacteria and the like passing through the implant and into the interior of the patient's eye, for instance into the anterior chamber.

### BRIEF SUMMARY OF THE INVENTION

A first aspect of the present invention is embodied by a MEMS filter module having a first film or plate, a second film or plate, and a plurality of filtering walls. The first plate includes a plurality of first flow ports, while the second plate includes a plurality of second flow ports. Each filter wall extends along at least part of one of a plurality of at least generally concentrically disposed filtering wall reference circles. At least one filtering wall is associated with each filtering wall reference circle. Each filtering wall extends from the second plate at least generally toward the first plate such that a space between the first plate and each filtering wall defines a filter trap (e.g., an area to “trap” particulates, certain-sized cells, or the like).

Various refinements exist of the features noted in relation to the first aspect of the present invention. Further features may also be incorporated in the first aspect of the present invention as well. These refinements and additional features may exist individually or in any combination. One embodiment has each filtering wall terminating prior to reaching the first plate. Another embodiment has each filtering wall extending within a first flow port of the first plate, but so as to remain spaced from the first plate to define a filter trap. Any way of defining a filter trap between the first plate and each filtering wall may be utilized.

Each filtering wall reference circle could include a single filtering wall (e.g., an “annular” filtering wall in accordance with the following) or a plurality of filtering walls that are appropriately spaced from each other. Adjacent filtering walls that are disposed along a common filtering wall reference circle could be separated by the same size of gap that exists between each filtering wall and the first plate. One or more of the filtering walls also may be annular, including having each filtering wall of the MEMS filter module be annular such that the plurality of filtering walls would be at least generally concentrically disposed. “Annular” or the like herein means that the relevant structure extends a full 360° about a certain reference point or axis, and does not limit the relevant structure to a circular configuration. Representative annular filtering walls would include circular, square-shaped, rectangular-shaped, elliptical-shaped, or the like. Circular annular filtering walls are preferred since they are believed to accommodate a desirably high flow rate through the MEMS filter module, or otherwise provide one or more desired flow characteristics. The annular filtering walls may be equally spaced, although such is not required.

The first and second flow ports preferably extend completely through the first and second plates, respectively, and may be of any appropriate size, shape, and/or configuration. Each first flow port may be disposed on one of a plurality of at least generally concentrically disposed first reference circles, and each first reference circle may have at least one associated first flow port. Each first reference circle should be offset from each filtering wall reference circle in a lateral dimension (“lateral” or “radially” meaning in a direction that is at least generally perpendicular to a thickness dimension of the first and second plates) such that each first flow port is laterally offset from each filtering wall. As such, a flow through the MEMS filter module would have at least a certain lateral component in order to progress between a first flow port and any associated filter trap.

Each second flow port may be disposed on one of a plurality of at least generally concentrically disposed second reference circles, and each second reference circle may have at least one associated second flow port. Each second reference circle should be offset from each filtering wall reference circle in the lateral dimension such that each second flow port is laterally offset from each filtering wall. As such, a flow through the MEMS filter module would have at least a certain lateral component in order to progress between a second flow port and any associated filter trap. In one embodiment, the first and second flow ports are associated with first and second reference circles, respectively, in the above noted manner, each first flow port is laterally offset from each second flow port and is also laterally offset from each filtering wall, and each second flow port is also laterally offset from each filtering wall.

A space at least somewhere between each adjacent pair of filtering wall reference circles may include a chamber (e.g., a space between the filtering walls of adjacent filtering wall reference circles). A first member of each adjacent pair of chambers (i.e., one of the chambers in a given pair) may be associated with only a first flow port type, while a second member of each adjacent pair of chambers (i.e., the other chamber in a given pair) may be associated with only a second flow port type. The plurality of first flow ports may be of the first flow port type, while the plurality of second flow ports may be of the second flow port type. One embodiment has each chamber being associated with either a plurality of first flow ports or a plurality of second flow ports. Another embodiment has each chamber being associated with at least one first flow port, but no second flow ports, or being associated with at least one second flow port, but no first flow ports.

The MEMS filter module may include what may be characterized as a plurality of first flow port chambers and a plurality of second flow port chambers. Each first flow port chamber and each second flow port chamber may be bounded in one dimension by the first and second plates, and may be bounded in another dimension by each filtering wall associated with adjacent pairs of filtering wall reference circles. Therefore, the first and second flow port chambers may be characterized as being at least generally annular.

The above-noted first and second flow port chambers may be arranged such that they are disposed in alternating relation. That is, one first flow port chamber may be disposed between each adjacent pair of second flow port chambers, and one second flow port chamber may be disposed between each adjacent pair of first flow port chambers. Each first flow port chamber is associated with only a first flow port type, while each second flow port chamber is associated with only a second flow port type. In accordance with the foregoing, the plurality of first flow ports may be of the first flow port type, while the plurality of second flow ports may be of the second flow port type.

A second aspect of the present invention is embodied by a MEMS filter module having a first film or plate, a second film or plate, and a plurality of annular filtering walls that are disposed about a common point (i.e., each filtering wall extends completely about any adjacent filtering wall that is disposed inwardly thereof). The first plate includes a plurality of first flow ports, while the second plate includes a plurality of second flow ports. Each filtering wall extends from the second plate at least generally toward the first plate such that a space between the first plate and each filtering wall defines a filter trap (e.g., an area to “trap” particulates, certain-sized cells, or the like).

Various refinements exist of the features noted in relation to the second aspect of the present invention. Further features may also be incorporated in the second aspect of the present invention as well. These refinements and additional features may exist individually or in any combination. One embodiment has each filtering wall terminating prior to reaching the first plate. Another embodiment has each filtering wall extending within a first flow port of the first plate, but so as to remain spaced from the first plate to define a filter trap. Any way of defining a filter trap between the first plate and each filtering wall may be utilized.

“Annular” again means that each filtering wall extends a full 360° about a certain reference point or axis, and does not limit the filtering walls to a circular configuration. Representative annular filtering walls would include circular, square-shaped, rectangular-shaped, elliptical-shaped, or the like. Circular annular filtering walls are preferred since they are believed to accommodate a desirably high flow rate through the MEMS filter module, or otherwise provide one or more desired flow characteristics. The annular filtering walls may be equally spaced, although such is not required. Moreover, the annular filtering walls may be at least generally concentrically disposed, although such is not required.

The first and second flow ports preferably extend completely through the first and second plates, respectively, and may be of any appropriate size, shape, and/or configuration. Each first flow port may be disposed on one of a plurality of at least generally concentrically disposed first reference circles, and each first reference circle may have at least one associated first flow port. Each first reference circle should be laterally offset from each filtering wall such that each first flow port is laterally offset from each filtering wall. As such, a flow through the MEMS filter module would have at least a certain lateral component in order to progress between a first flow port and any associated filter trap (“lateral” again meaning in a direction that is at least generally perpendicular to a thickness dimension of the first and second plates).

Each second flow port may be disposed on one of a plurality of at least generally concentrically disposed second reference circles, and each second reference circle may have at least one associated second flow port. Each second reference circle should be laterally offset from each filtering wall such that each second flow port is laterally offset from each filtering wall. As such, a flow through the MEMS filter module would have at least a certain lateral component in order to progress between a second flow port and any associated filter trap. In one embodiment, the first and second flow ports are associated with first and second reference circles, respectively, in the above noted manner, each first flow port is laterally offset from each second flow port and is laterally offset from each filtering wall, and each second flow port is also laterally offset from each filtering wall.

The space between each adjacent pair of annular filtering walls may be characterized as a chamber. A first member of each adjacent pair of chambers (i.e., one of the chambers in a given pair) may be associated with only a first flow port type, while a second member of each adjacent pair of chambers (i.e., the other chamber in a given pair) may be associated with only a second flow port type. The plurality of first flow ports may be of the first flow port type, while the plurality of second flow ports may be of the second flow port type. One embodiment has each chamber being associated with either a plurality of first flow ports or a plurality of second flow ports. Another embodiment has each chamber being associated with at least one first flow port, but no second flow ports, or being associated with at least one second flow port, but no first flow ports.

The MEMS filter module may include what may be characterized as a plurality of first flow port chambers and a plurality of second flow port chambers. Each first flow port chamber and each second flow port chamber may be bounded in one dimension by the first and second plates, and may be bounded in another dimension by pairs of adjacent annular filtering walls. Therefore, the first and second flow port chambers may be characterized as being annular.

The above-noted first and second flow port chambers may be arranged such that they are disposed in alternating relation. That is, one first flow port chamber may be disposed between each adjacent pair of second flow port chambers, and one second flow port chamber may be disposed between each adjacent pair of first flow port chambers. Each first flow port chamber is associated with only a first flow port type, while each second flow port chamber is associated with only a second flow port type. In accordance with the foregoing, the plurality of first flow ports may be of the first flow port type, while the plurality of second flow ports may be of the second flow port type.

A plurality of structural interconnects may extend between the first and second plates so as to maintain the same in an at least substantially fixed position relative to each other in the case of the MEMS filter modules described herein. These interconnects may be of any appropriate size, shape, and/or configuration, and may be disposed in any appropriate arrangement. In one embodiment, a plurality of columns, posts, or the like extend between and interconnect the first and second plates somewhere in the space between adjacent filtering walls in the lateral or radial dimension. Consider the case where the filtering walls are annular. Appropriate structural interconnects may extend between the first and second plates at a location that is between each adjacent pair of filtering walls. Although structural interconnects that are equidistantly disposed from a common reference point may be equally spaced from each other, such is not necessarily required.

One or more annular structural interconnects may extend between the first and second plates in case of the MEMS filter modules described herein. Providing multiple, laterally or radially spaced annular structural interconnects toward an outer perimeter of a particular MEMS filter module (“perimeter annular structural interconnects”) provides redundant radial seals of sorts. That is, these types of perimeter annular structural interconnects may reduce the potential for a fluid flowing out from between the first and second plates. These types of perimeter annular structural interconnects also potentially enhance the rigidity of the MEMS filter module. Preferably, the various first flow ports, second flow ports, and filtering walls would be disposed or located inwardly of each such perimeter annular structural interconnect. One or more additional annular structural interconnects could be utilized at other locations, for instance to reinforce the MEMS filter module.

Any of the MEMS filter modules described herein may be disposed in a flow path of any appropriate type (e.g., between a pair of sources of any appropriate type, such as a man-made reservoir, a biological reservoir, and/or the environment), and further may be used for any appropriate application. That is, one or more of any of these MEMS filter modules could be disposed in a conduit that fluidly interconnects multiple sources (e.g., two or more), and each source may be either a man-made reservoir, a biological reservoir, the environment, or any other appropriate source. One example would be to dispose one or more of these MEMS filter modules in a conduit extending between the anterior chamber of an eye and a location that is exterior of the cornea of the eye. Another example would be to dispose one or more of these MEMS filter modules in a conduit extending between the anterior chamber of an eye and another location that is exterior of the sclera of the eye. Yet another example would be to dispose one or more of these MEMS filter modules in a conduit extending between the anterior chamber of an eye and another location within the eye (e.g., into Schlemm's canal) or body. In any case, any of these MEMS filter modules could be disposed directly into such a conduit, or one or more housings could be used to integrate any of these MEMS filter modules with the conduit. In each of these examples, the conduit would provide an exit path for aqueous humor when installed for a glaucoma patient. That is, each of these examples may be viewed as a way of treating glaucoma or providing at least some degree of control of the intraocular pressure.

Each of the MEMS filter modules described herein may be used in combination with a conduit to define an implant that is installable in a biological mass. In this regard, the conduit may include a flow path that is adapted to fluidly interconnect with a first body region, and at least one MEMS filter module may be disposed within this flow path. In one embodiment, at least one housing is used to establish an interconnection or interface between the conduit and the MEMS filter module. For instance, the housing may be at least partially disposed within the conduit, and the MEMS filter module may interface with the housing. Although any appropriate implant application is contemplated, in one embodiment the implant is installable in a human eye to fluidly interconnect with an anterior chamber of the human eye for purposes of regulating intraocular pressure.

Surface micromachining is the preferred technology for fabricating the MEMS filter modules described herein. In this regard, the first and second plates of the MEMS filter modules described herein each may be fabricated from one or more layers or films, where each layer or film has a thickness of no more than about 10 microns in one embodiment, and more typically a thickness within a range of about 1 micron to about 3 microns in another embodiment. Each of the MEMS filter modules described herein may be fabricated in at least two different fabrication levels that are spaced from each other (hereafter a first fabrication level and a second fabrication level). “Fabrication level” corresponds with what may be formed by a deposition of a structural material before having to form any overlying layer of a sacrificial material (e.g., from a single deposition of a structural layer or film). The first plate may be fabricated at least in the first fabrication level, while the second plate may be fabricated in at least the second fabrication level. It should be appreciated that the characterization of the first plate being in the “first fabrication level” and the second plate being in the “second fabrication level” by no means requires that the first fabrication level be that which is deposited “first”, and that the second fabrication level be that which is deposited “second.” Moreover, it does not require that the first fabrication level and the second fabrication level be immediately adjacent to each other. These MEMS filter modules may be fabricated on an appropriate substrate and where the first plate is fabricated in one structural layer that is disposed somewhere between the substrate and another structural layer in which the second plate is fabricated, or vice versa.

The first and second plates each may exist in a single fabrication level or may exist in multiple fabrication levels. In the above-noted first instance, a deposition of a structural material in a single fabrication level may define an at least generally planar layer. Another option regarding the first instance would be for the deposition of a structural material in a single fabrication level to define an at least generally planar portion, plus one or more structures that extend down toward, but not to, the underlying structural layer at the underlying fabrication level. For instance, the second plate and the plurality of filtering walls could be fabricated in a common fabrication level that is different than the fabrication level associated with the first plate. In either situation and prior to the release, in at least some cases there will be at least some thickness of sacrificial material disposed between the first and second plates prior to the release. Similarly, prior to the release there will be sacrificial material between the end of the filtering walls and the first plate. Removal of this particular sacrificial material by the release will thereby define the noted filter traps.

In the above-noted second instance, two or more structural layers or films from adjacent fabrication levels could be disposed in direct interfacing relation (e.g., one directly on the other). Over the region that is to define the first plate or second plate, this would require removal of at least some of the sacrificial material that is deposited on the structural material at one fabrication level before depositing the structural material at the next fabrication level (e.g. sacrificial material may be encased by a structural material, so as to not be removed by the release). Another option regarding the above-noted second instance would be to maintain the separation between structural layers or films in different fabrication levels for the first plate and second plate, but provide an appropriate structural interconnection therebetween (e.g., a plurality of columns, posts, or the like extending between adjacent structural layers or films in different, spaced fabrication levels). For instance, the second plate and the various structural interconnects may be in a common fabrication level, and the first plate may be fabricated in a different fabrication level. Alternatively, the first plate and the various structural interconnects may exist in one fabrication level, while the second plate exists in a different fabrication level.

### BRIEF DESCRIPTION OF THE SEVERAL VIEWS OF THE DRAWING

FIG. 1 is a side view of a plurality of layers that may be used by one embodiment of a surface micromachining fabrication technique.

FIG. 2 is a perspective view of one embodiment of a MEMS filter module that utilizes a plurality of concentrically disposed, annular filter walls.

FIG. 3 is a cross-sectional, exploded, perspective view of the MEMS filter module of FIG. 2, taken along a plane between its first and second plates so as to not intersect with the various annular filter walls that extend from the second plate toward, but not to, the first plate, and with the second plate having been pivoted away from the first plate.

FIG. 4A is an enlarged, cross-sectional view that illustrates part of the first plate of the MEMS filter module of FIG. 2, where the cross-section is taken along a plane that extends between its first and second plates so as to intersect with the annular filter walls that extend from the second plate toward, but not to, the first plate.

FIG. 4B is a plan view of part of the first plate of the MEMS filter module of FIG. 2, illustrating a plurality of concentrically disposed reference circles along which its first flow ports are disposed and a plurality of concentrically disposed reference circles coinciding with the location of the filtering walls of the second plate.

FIG. 5A is an enlarged, cross-sectional view that illustrates part of the second plate of the MEMS filter module of FIG. 2, where the cross-section is taken along a plane between its first and second plates so as to not intersect with the annular filter walls that extend from the second plate toward, but not to, the first plate.

FIG. 5B is a plan view of part of the second plate of the MEMS filter module of FIG. 2, illustrating a plurality of concentrically disposed reference circles along which its second flow ports and a plurality of concentrically disposed reference circles along which the filtering walls are disposed.

FIG. 6 is a cross-sectional view that illustrates the spacing between the first plate and the annular filtering walls of the second plate in the case of the MEMS filter module of FIG. 2.

FIG. 7 is a cross-sectional, exploded, perspective view of the MEMS filter module of FIG. 2, taken along a plane that extends between its second plate and an annular support or ring that sandwiches the second plate between this annular support and the first plate, with this annular support having been pivoted away from the second plate.

FIG. 8A is an exploded, perspective view of one embodiment of a flow assembly that uses a MEMS flow module.

FIG. 8B is a perspective view of the flow assembly of FIG. 8A in an assembled condition.

FIG. 9A is an exploded, perspective of another embodiment of a flow assembly that uses a MEMS flow module.

FIG. 9B is a perspective view of the flow assembly of FIG. 9A in an assembled condition.

FIG. 10A is an exploded, perspective of another embodiment of a flow assembly that uses a MEMS flow module.

FIG. 10B is a perspective view of the flow assembly of FIG. 10A in an assembled condition.

FIG. 11A is a schematic of one embodiment of a glaucoma or intraocular implant that may use any of the MEMS flow modules described herein.

FIG. 11B is a cross-sectional view of one embodiment of glaucoma or intraocular implant or shunt that is used to relieve pressure within the anterior chamber of the eye, and that may utilize any of the MEMS flow modules described herein.

### DETAILED DESCRIPTION OF THE INVENTION

The present invention will now be described in relation to the accompanying drawings that at least assist in illustrating its various pertinent features. Generally, the devices described herein are microfabricated. There are a number of microfabrication technologies that are commonly characterized as “micromachining,” including without limitation LIGA (Lithographie, Galvonoformung, Abformung), SLIGA (sacrificial LIGA), bulk micromachining, surface micromachining, micro electrodischarge machining (EDM), laser micromachining, 3-D stereolithography, and other techniques. Hereafter, the term “MEMS device,” “microfabricated device,” or the like means any such device that is fabricated using a technology that allows realization of a feature size of 10 microns or less. Any appropriate microfabrication technology or combination of microfabrication technologies may be used to fabricate the various devices to be described herein.

Surface micromachining is currently the preferred fabrication technique for the various devices to be described herein. One particularly desirable surface micromachining technique is described in U.S. Pat. No. 6,082,208, that issued Jul. 4, 2000, that is entitled “Method For Fabricating Five-Level Microelectromechanical Structures and Microelectromechanical Transmission Formed,” and the entire disclosure of which is incorporated by reference in its entirety herein. Surface micromachining generally entails depositing alternate layers of structural material and sacrificial material using an appropriate substrate (e.g., a silicon wafer) which functions as the foundation for the resulting microstructure. Various patterning operations (collectively including masking, etching, and mask removal operations) may be executed on one or more of these layers before the next layer is deposited so as to define the desired microstructure. After the microstructure has been defined in this general manner, all or a portion of the various sacrificial layers are removed by exposing the microstructure and the various sacrificial layers to one or more etchants. This is commonly called “releasing” the microstructure.

The term “sacrificial layer” as used herein means any layer or portion thereof of any surface micromachined microstructure that is used to fabricate the microstructure, but which does not generally exist in the final configuration (e.g., sacrificial material may be encased by a structural material at one or more locations for one or more purposes, and as a result this encased sacrificial material is not removed by the release). Exemplary materials for the sacrificial layers described herein include undoped silicon dioxide or silicon oxide, and doped silicon dioxide or silicon oxide (“doped” indicating that additional elemental materials are added to the film during or after deposition). The term “structural layer” as used herein means any other layer or portion thereof of a surface micromachined microstructure other than a sacrificial layer and a substrate on which the microstructure is being fabricated. Exemplary materials for the structural layers described herein include doped or undoped polysilicon and doped or undoped silicon. Exemplary materials for the substrates described herein include silicon. The various layers described herein may be formed/deposited by techniques such as chemical vapor deposition (CVD) and including low-pressure CVD (LPCVD), atmospheric-pressure CVD (APCVD), and plasma-enhanced CVD (PECVD), thermal oxidation processes, and physical vapor deposition (PVD) and including evaporative PVD and sputtering PVD, as examples.

In more general terms, surface micromachining can be done with any suitable system of a substrate, sacrificial film(s) or layer(s) and structural film(s) or layer(s). Many substrate materials may be used in surface micromachining operations, although the tendency is to use silicon wafers because of their ubiquitous presence and availability. The substrate is essentially a foundation on which the microstructures are fabricated. This foundation material must be stable to the processes that are being used to define the microstructure(s) and cannot adversely affect the processing of the sacrificial/structural films that are being used to define the microstructure(s). With regard to the sacrificial and structural films, the primary differentiating factor is a selectivity difference between the sacrificial and structural films to the desired/required release etchant(s). This selectivity ratio may be on the order of about 10:1, and is more preferably several hundred to one or much greater, with an infinite selectivity ratio being most preferred. Examples of such a sacrificial film/structural film system include: various silicon oxides/various forms of silicon; poly germanium/poly germanium-silicon; various polymeric films/various metal films (e.g., photoresist/aluminum); various metals/various metals (e.g., aluminum/nickel); polysilicon/silicon carbide; silicone dioxide/polysilicon (i.e., using a different release etchant like potassium hydroxide, for example). Examples of release etchants for silicon dioxide and silicon oxide sacrificial materials are typically hydrofluoric (HF) acid based (e.g., concentrated HF acid, which is actually 49 wt % HF acid and 51 wt % water; concentrated HF acid further diluted with water; buffered HF acid (HF acid and ammonium fluoride)).

The microfabrication technology described in the above-noted '208 Patent uses a plurality of alternating structural layers (e.g., polysilicon and therefore referred to as “P” layers herein) and sacrificial layers (e.g., silicon dioxide, and therefore referred to as “S” layers herein). The nomenclature that is commonly used to describe the various layers in the microfabrication technology described in the above-noted '208 Patent will also be used herein.

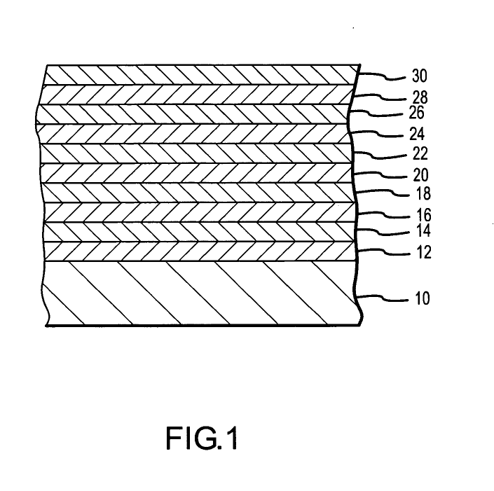

FIG. 1 generally illustrates one embodiment of layers on a substrate 10 that is appropriate for surface micromachining and in accordance with the nomenclature commonly associated with the '208 Patent. Each of these layers will typically have a thickness of no more than about 10 microns, and more typically a thickness within a range of about 1 micron to about 3 microns. Progressing away from the substrate 10, the various layers are: a dielectric layer 12 (there may be an intermediate oxide layer between the dielectric layer 12 and the substrate 10 as well, which is not shown); a P₀ layer 14 (a first fabrication level); an S₁ layer 16; a P₁ layer 18 (a second fabrication level); an S₂ layer 20; a P₂ layer 22 (a third fabrication level); an S₃ layer 24; a P₃ layer 26 (a fourth fabrication level); an S₄ layer 28; and a P₄ layer 30 (a fifth fabrication level). In some cases, the S₂ layer 20 may be removed before the release such that the P₂ layer 22 is deposited directly on the P₁ layer 18, and such will hereafter be referred to as a P₁/P₂ layer. It should also be appreciated that one or more other layers may be deposited on the P₄ layer 30 after the formation thereof and prior to the release, where the entirety of the S₁ layer 16, S₂ layer 20, S₃ layer 24, and S₄ layer 28 may be removed (although portions of one or more of these layers may be retained for one or more purposes if properly encased so as to be protected from the release etchant). It should also be appreciated that adjacent structural layers may be structurally interconnected by forming cuts or apertures through the entire thickness of a particular sacrificial layer before depositing the next structural layer. In this case, the structural material will not only be deposited on the upper surface of the particular sacrificial layer, but will be deposited in these cuts or apertures as well (and will thereby interconnect a pair of adjacent, spaced, structural layers).

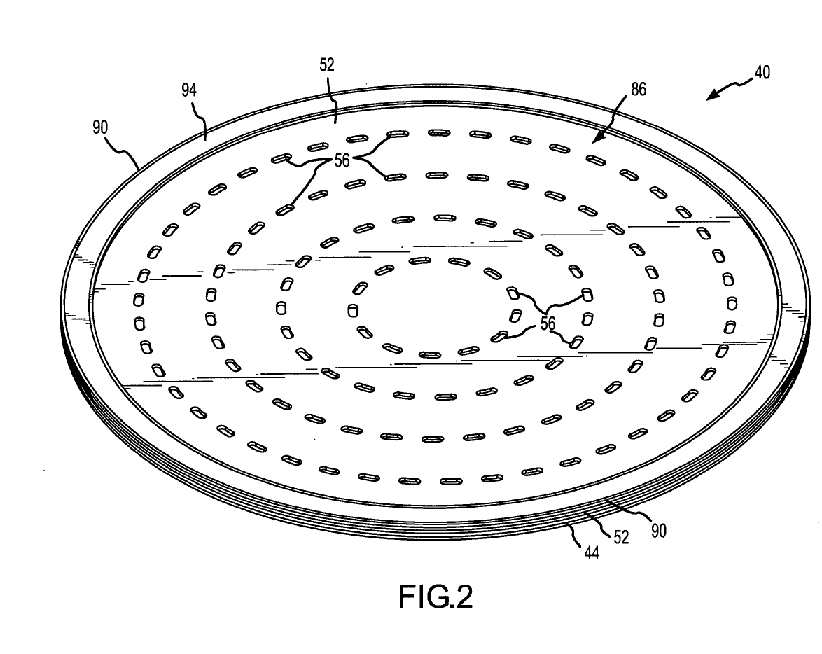

One embodiment of a MEMS filter module is illustrated in FIGS. 2-7, may be fabricated at least generally in accordance with the above-noted discussion of FIG. 1, and is identified by reference numeral 40. Components of the MEMS filter module 40 include a first plate 44 (e.g., fabricated in at least P₂ layer 22; fabricated in a combination of P₂ layer 22 that is disposed directly on P₁ layer 18), a second plate 52 (e.g., fabricated in P₃ layer 26), and a plurality of filtering walls 60 (e.g., fabricated in P₃ layer 26). The first plate 44 includes a plurality of first flow ports 48 that extend completely through the first plate 44, while the second plate 52 includes a plurality of second flow ports 56 that extend completely through the second plate 52. Both the first flow ports 48 and the second flow ports 56 may be of any appropriate size, shape, and/or configuration.

The first plate 44 and the second plate 52 of the MEMS filter module 40 may be maintained in at least a substantially fixed position relative to each other. In this regard, a plurality of structural interconnects 76 extend between and structurally interconnect the first plate 44 and the second plate 52 so as to maintain the same in spaced relation. Each structural interconnect 76 may be of any appropriate size, shape, and/or configuration (e.g., in the form of a column or post, as shown), and the plurality of structural interconnects 76 may be disposed in any appropriate arrangement.

Perimeter portions of the first plate 44 and the second plate 52 also may be structurally interconnected by one or more annular structural interconnects 82. “Annular” only means that the structural interconnects 82 extend a full 360° about a common point, and does not limit the annular structural interconnects 82 to having a circular configuration. Representative annular configurations for the annular structural interconnects 82 include circular, square-shaped, rectangular-shaped, elliptical-shaped or the like. Each annular structural interconnect 82 also provides a lateral or radial seal function by reducing the potential for a flow exiting the MEMS filter module 40 from the space between the first plate 44 and the second plate 52. Utilizing multiple, laterally or radially-spaced annular structural interconnects 82 thereby provides redundant lateral or radial seals.

The MEMS filter module 40 accommodates filtering of a bidirectional flow, or a flow through the MEMS filter module 40 in either of two general directions. That is, a flow may be directed into the MEMS filter module 40 through one or more of the first flow ports 48 of the first plate 44 and may exit the MEMS filter module 40 through one or more of the second flow ports 56 of the second plate 52. A flow may also be directed into the MEMS filter module 40 through one or more of the second flow ports 56 of the second plate 52 and may exit the MEMS filter module 40 through one or more of the first flow ports 48 of the first plate 44. Regardless of the direction of flow through the MEMS filter module 40, this flow is filtered by the first plate 44 cooperating with a plurality of the filtering walls 60 of the MEMS filter module 40.

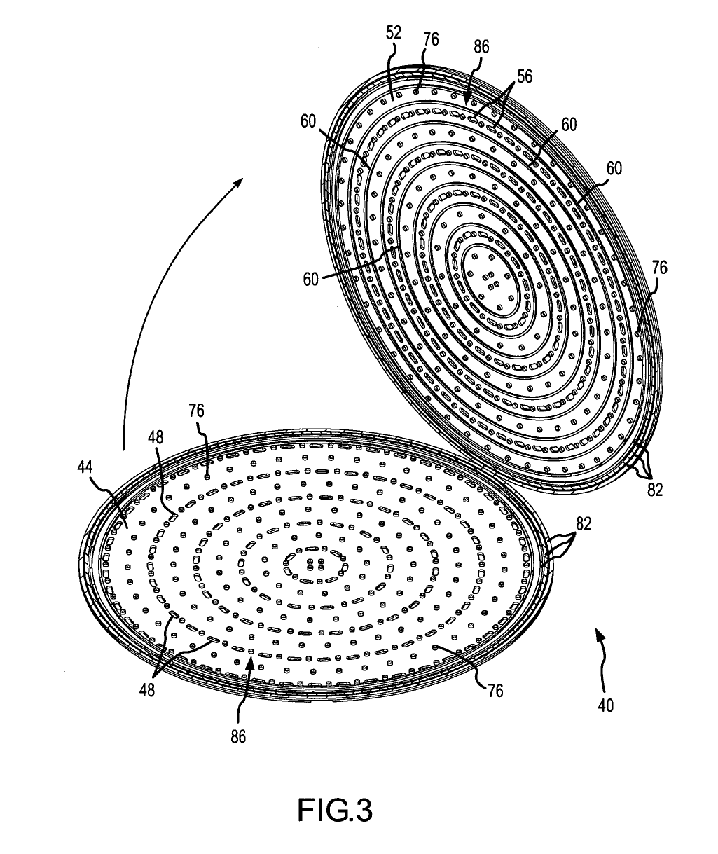

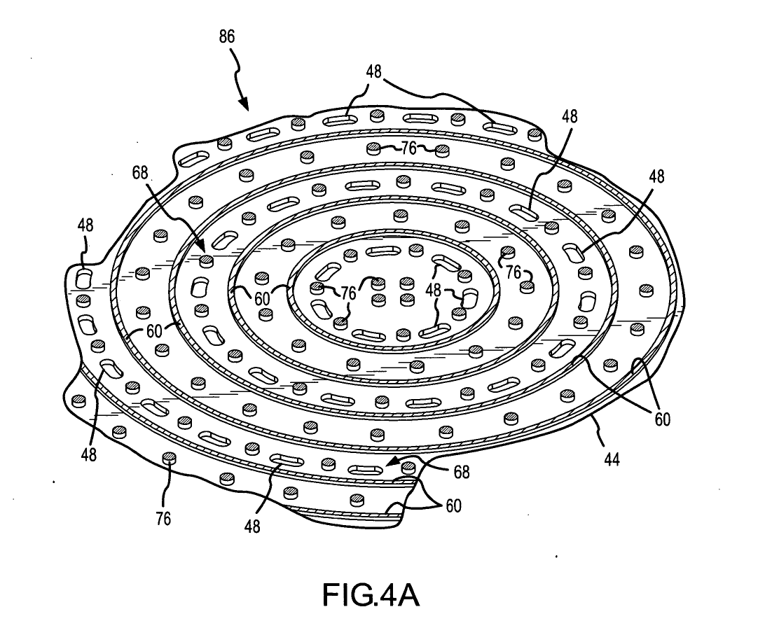

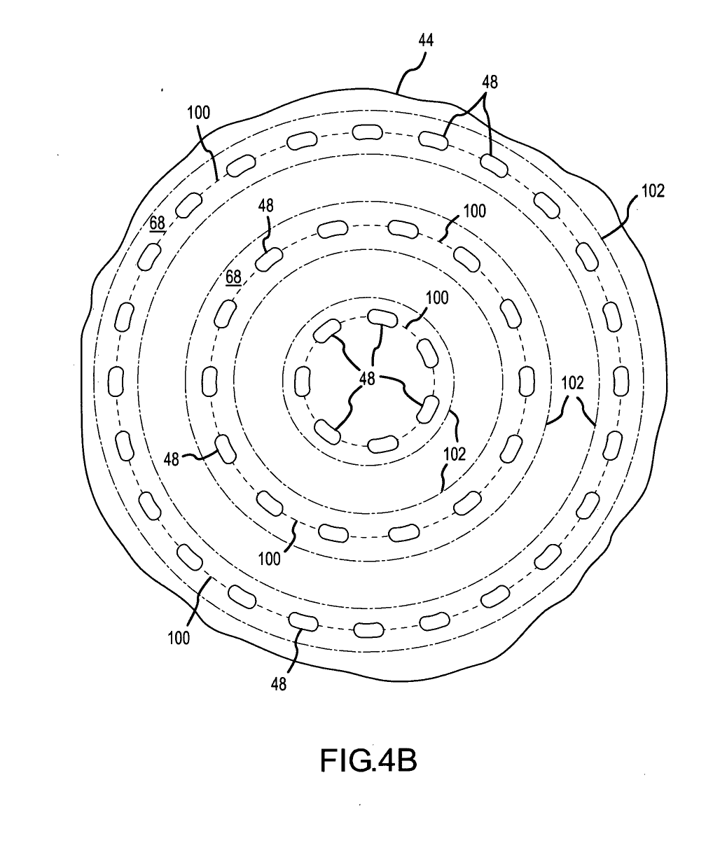

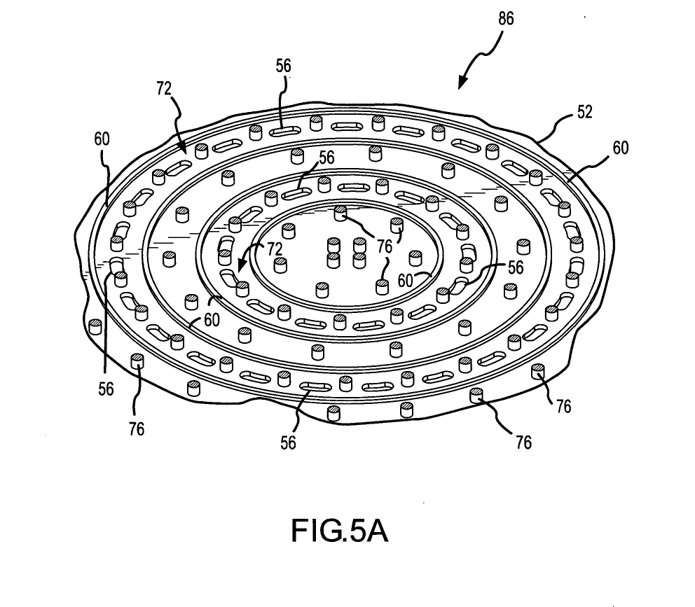

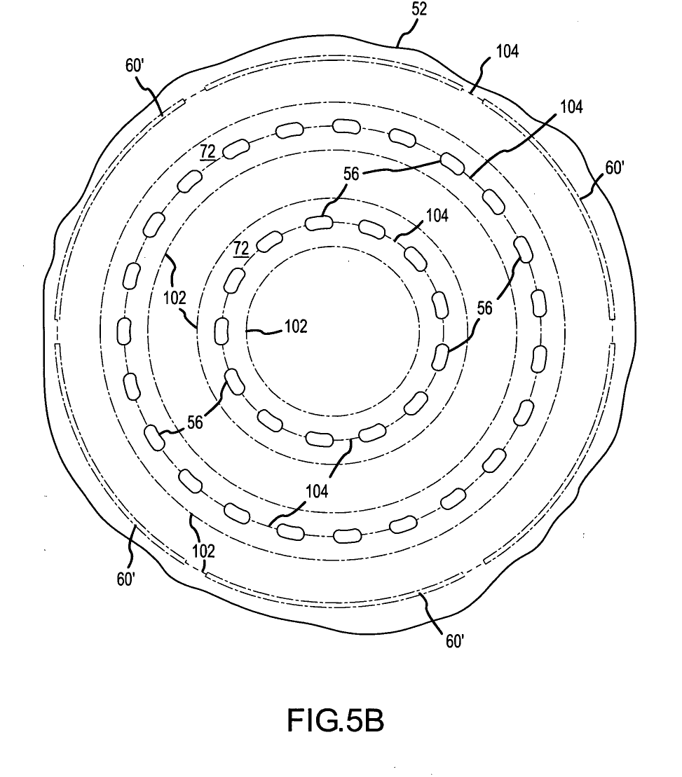

As illustrated in FIGS. 3-6, each filtering wall 60 extends from the second plate 52 toward, but not to, the first plate 44 (the cross-sectional view in FIG. 3 is taken between the first plate 44 and the second plate 52 along a reference plane that extends through the space between the first plate 44 and the end of each of the filtering walls 60; the cross-sectional view in FIG. 4A is taken between the first plate 44 and the second plate 52 along a reference plane that intersects with the various filtering walls 60; the cross-sectional view in FIG. 5A is taken between the first plate 44 and the second plate 52 along a reference plane that extends through the space between the first plate 44 and the end of each of the filtering walls 60). That is, each filtering wall 60 terminates prior to reaching the first plate 44. Any appropriate number of filtering walls 60 may be utilized by the MEMS filter module 40.

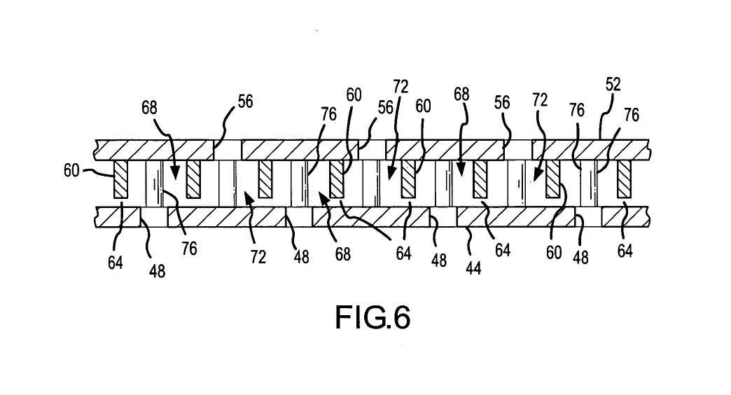

The space between the end of each filtering wall 60 and the first plate 44 is identified as a filter trap 64 (FIG. 6) and is dimensioned so as to filter objects of a certain size or larger. The height of each filter trap 64 (the distance between the end of each filtering wall 60 and the first plate 44) is no more than about 0.4 microns in one embodiment, is about 0.2 to about 0.3 microns in one embodiment, and is no more than about 0.1 microns in yet another embodiment. The size of each filter trap 64 may be established at any appropriate value. Although each filter trap 64 may be of at least substantially the same size, such need not always be the case.

Each filtering wall 60 is annular in that each filtering wall 60 extends a full 360° about a reference point, as illustrated in FIGS. 3-5A. Although a circular annular configuration is preferred for the filtering walls 60, other annular configurations could be utilized as well (e.g., square-shaped, rectangular-shaped, elliptical-shaped). In addition, one or more filtering walls 60 could be replaced by a plurality of appropriately spaced filtering wall segments 60′ as illustrated by the dashed line in FIG. 5B. In any case, the filtering walls 60 are disposed in a desired arrangement that is believed to accommodate a desirably high flow rate through the MEMS filter module 40. In this regard, the various filtering walls 60 are at least generally concentrically disposed about a common center or point (i.e., each being disposed at a different radius from a common center or point). Although it is preferred for the filtering walls 60 to be equally spaced in this concentric arrangement, such is not necessarily required.

The various first flow ports 48, the various second flow ports 56, and the various filtering walls 60 are located in what may be characterized as a filtering region 86 of the MEMS filter module 40. The filtering region 86 is located inwardly of the innermost annular structural interconnect 82 between the first plate 44 and the second plate 52. Generally, the first flow ports 48 through the first plate 44 and the second flow ports 56 through the second plate 52 are arranged such that: 1) any flow entering the MEMS filter module 40 through any first flow port 48 will flow through a filter trap 64 prior to exiting the MEMS filter module 40 through any second flow port 56; and 2) any flow entering the MEMS filter module 40 through any second flow port 56 will flow through a filter trap 64 prior to exiting the MEMS filter module 40 through any first flow port 48.

The space between each adjacent pair of filtering walls 60 is accessed by either one or more first flow ports 48 or one or more second flow ports 56 in order to force a flow through at least one filter trap 64 in the case of the MEMS filter module 40. Stated another way, the MEMS filter module 40 may be characterized as including a plurality of first flow port chambers 68 and a plurality of the second flow port chambers 72 (e.g., FIG. 6). Each first flow port chamber 68 and each second flow port chamber 72 is defined by a spacing between the first plate 44 and the second plate 52 in a first dimension, and is defined by a spacing between adjacent filtering walls 60 in a second dimension that is orthogonal to the first dimension. Only first flow ports 48 directly fluidly communicate with each first flow port chamber 68—no second flow port 56 can access a first flow port chamber 68 without first passing through a filter trap 64. Similarly, only second flow ports 56 directly fluidly communicate with each second flow port chamber 72—no first flow port 48 can access a second flow port chamber 72 without first passing through a filter trap 64.

The above-noted first flow port chambers 68 and the second flow port chambers 72 are disposed in alternating relation to force all flow through at least one filter trap 64. For instance, a flow entering the MEMS filter module 40 through one or more first flow ports 48 of a particular first flow port chamber 68 would need to flow through at least one filter trap 64 before entering any second flow port chamber 72, such that the flow could then exit the MEMS filter module 40 through one or more second flow ports 56 associated with this particular second flow port chamber 72. Similarly, a flow entering the MEMS filter module 40 through one or more second flow ports 56 of a particular second flow port chamber 72 would need to flow through at least one filter trap 64 before entering any first flow port chamber 68, such that the flow could then exit the MEMS filter module 40 through one or more first flow ports 48 associated with this particular first flow port chamber 68.

Another way to characterize the arrangement of the first flow ports 48 of the first plate 44, the second flow ports 56 of the second plate 52, and the filter walls 60 of the second plate 52 is that they are each located or disposed on a plurality of concentrically disposed reference circles. FIG. 4B illustrates that a plurality of first flow ports 48 for the first plate 44 are disposed on each of a plurality of reference circles 100. Also shown in FIG. 4B are the reference circles 102 that identify the location of the filtering walls 60 that extend from the second plate 52, and that cooperate with the first plate 44 to define the above-noted filter traps 64. FIG. 5B illustrates that a plurality of second flow ports 56 are disposed on each of a plurality of reference circles 104. Also shown in FIG. 5B are the reference circles 102 that identify the location of the filtering walls 60 that extend from the second plate 52.

FIG. 5B also illustrates that one or more of the annular filtering walls 60 could be replaced with a plurality of filtering wall segments 60′ that are disposed along the corresponding reference circle 102 and that are appropriately spaced from each other. In one embodiment, the spacing between adjacent pairs of any such filtering wall segments 60′ on a common reference circle 102 is in accordance with the discussion presented above with regard to the size of the filter traps 64. Although each of these filtering wall segments 60′ are shown as being of the same arc length, such need not be the case. Moreover, although the spacing between adjacent filtering wall segments 60′ on a given reference circle 102 is preferably equal, such need not be the case. It should also be noted that the spacing between adjacent filtering wall segments 60′ on one reference circle 102 need not be the same as the spacing between adjacent filtering wall segments 60′ on a different reference circle 102.

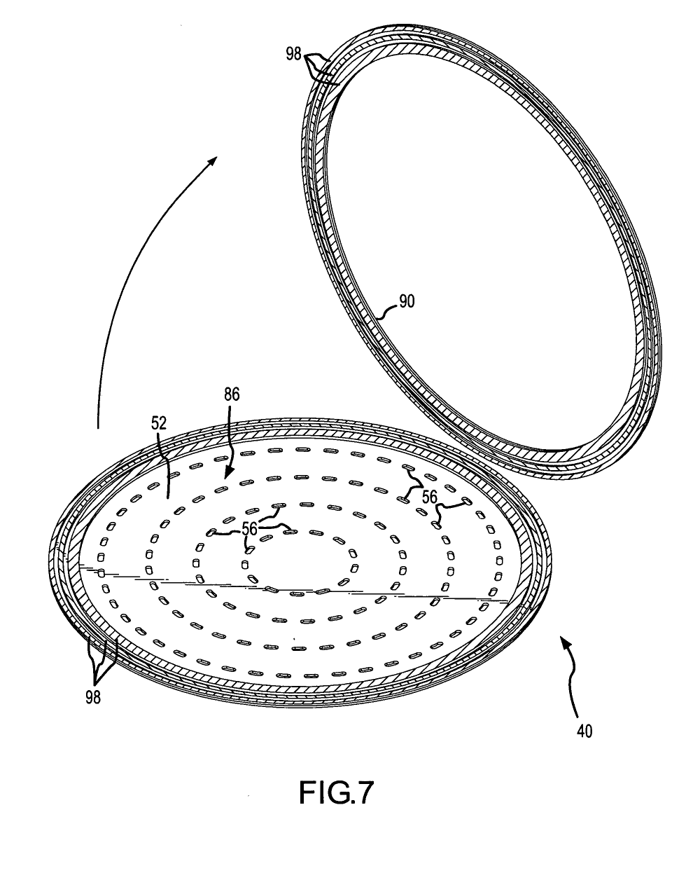

The MEMS filter module 40 could simply be in the form of the above-noted first plate 44, the second plate 52, and the filtering walls 60. However, it may be desirable to include one or more additional structures for one or more purposes. In this regard, the MEMS filter module 40 also may include an annular support 90 (e.g., fabricated in P₄ layer 30) that is spaced from and interconnected with a perimeter portion of the second plate 52 by a one or more annular structural interconnects 98 (FIGS. 2 and 7). Any appropriate number of annular structural interconnects 98 may be utilized. The second plate 52 is thereby “sandwiched” between the first plate 44 and the annular support 90. This configuration may enhance the rigidity of the MEMS filter module 40, or at least enhance an interface between the MEMS filter module 40 and one or more housings that may be utilized to contain/support the MEMS filter module 40 for a particular application. The annular support 90 could also be deposited directly on the second plate 52.

The MEMS filter module 40 may further include a ring 94 (FIG. 2) that is fixedly positioned on the surface of the annular support 90 that is opposite that which interfaces with the second plate 52. This ring 94 may be an appropriate metal that is attached to or formed on the annular support 90 after the MEMS filter module 40 has been fabricated, or may in fact be formed by surface micromachining as well (e.g., from another structural level). Generally, the ring 94 may provide a desired interface with a housing or other structure that incorporates the MEMS filter module 40. It should be appreciated that one or more additional plates with flow ports extending therethrough could be interconnected with or formed directly on either the first plate 44 or the second plate 52, and for any desired purpose.

The MEMS filter module 40 may be used for any appropriate application. One particularly desirable application is to use the MEMS filter module 40 in an implant that addresses the pressure in the anterior chamber of a patient's eye that is diseased. The size of the filter traps 64 may be selected to balance the desire to at least generally mimic the flow of aqueous humor out of the anterior chamber of a patient's eye through the eye's canal of Schlemm (e.g., provide a sufficient “back pressure”), along with the desire to be able to accommodate an increase in flow of aqueous humor out of the anterior chamber of the eye so relieve at least certain increases in the intraocular pressure in a desired manner.

Surface micromachining is the preferred technology for fabricating the above-described MEMS filter module 40. In this regard, the above-noted MEMS filter module 40 may be suspended above the substrate 10 after the release by one or more suspension tabs that are disposed about the perimeter of the MEMS filter module 40, that engage an appropriate portion of the MEMS filter module 40, and that are anchored to the substrate 10. These suspension tabs may be fractured or broken (e.g., by application of the mechanical force; electrically, such as by directing an appropriate current through the suspension tabs) to structurally disconnect the MEMS filter module 40 from the substrate 10. One or more motion limiters may be fabricated and disposed about the perimeter of the MEMS filter module 40 as well to limit the amount that the MEMS filter module 40 may move in the lateral or radial dimension after the suspension tabs have been fractured and prior to retrieving the disconnected MEMS filter module 40. Representative suspension tabs and motion limiters are disclosed in commonly owned U.S. patent application Ser. No. 11/048,195, that was filed on Feb. 1, 2005, that is entitled “MEMS FLOW MODULE WITH PIVOTING-TYPE BAFFLE,” and the entire disclosure of which is incorporated by reference herein.

The MEMS filter module 40 described herein may be fabricated in at least two different levels that are spaced from each other (hereafter a first fabrication level and a second fabrication level). Generally, that MEMS filter module 40 again includes the first plate 44 and the second plate 52 that are disposed in spaced relation, with a plurality of filtering walls 60 extending from the second plate 52 at least toward the first plate 44. The first plate 44 and its various first flow ports 48 may be fabricated in a first fabrication level, while the second plate 52 and its various second flow ports 56 and filtering walls 60 may be fabricated in a second fabrication level. It should be appreciated that the characterization of the first plate 44 being in a “first fabrication level” and the second plate 52 and filtering walls 60 being in the “second fabrication level” by no means requires that the first fabrication level be that which is deposited “first”, and that the second fabrication level be that which is deposited “second.” Moreover, it does not require that the first fabrication level and the second fabrication level be immediately adjacent.

One or both of the first plate 44 and that second plate 52 each may exist in a single fabrication level or may exist in multiple fabrication levels. “Fabrication level” corresponds with what may be formed by a deposition of a structural material before having to form any overlying layer of a sacrificial material (e.g., from a single deposition of a structural layer or film). In the above-noted first instance, a deposition of a structural material in a single fabrication level may define an at least generally planar layer. Another option regarding the first instance would be for the deposition of a structural material in a single fabrication level to define an at least generally planar portion, plus one or more structures that extend down toward, but not to, the underlying structural layer at the underlying fabrication level (e.g., the second plate 52 with the various filtering walls 60 extending downwardly therefrom, the fabrication of which is discussed in more detail below). In either situation and prior to the release, in at least some cases there will be at least some thickness of sacrificial material disposed between the entirety of the structures in adjacent fabrication levels (e.g., between the distal end of the filtering walls 60 and the first plate 44; between the first plate 44 and the second plate 52).

In the above-noted second instance, two or more structural layers or films from adjacent fabrication levels could be disposed in direct interfacing relation (e.g., one directly on the other). Over the region that is to define a pair of plates, this would require removal of at least some of the sacrificial material that is deposited on the structural material at one fabrication level before depositing the structural material at the next fabrication level (e.g., the annular support 90 could be deposited directly on a perimeter portion of the second plate 52, as previously noted). Another option regarding the above-noted second instance would be to maintain the separation between structural layers or films in different fabrication levels for a pair of plates, but provide an appropriate structural interconnection therebetween (e.g., a plurality of columns, posts, or the like extending between adjacent structural layers or films in different, spaced fabrication levels). For instance and as described above, the first plate 44 and the second plate 52 are disposed in spaced relation, but perimeter portions thereof are interconnected by the annular structural interconnects 82. The first plate 44 and the second plate 52 are also maintained in spaced relation by the structural interconnects 76 disposed within the filtering region 86. The structural interconnects 76, the annular structural interconnects 82, the second plate 52, and the filtering walls 60 may be fabricated in a common fabrication level.

With further regard to fabricating the MEMS filter module 40 at least in part by surface micromachining, each component thereof (including without limitation the first plate 44 and/or the second plate 52) may be fabricated in a structural layer or film at a single fabrication level (e.g., in P₁ layer 18; in P₂ layer 22; in P₃ layer 26; in P₄ layer 30 (FIG. 1 discussed above)). One example of fabricating the MEMS filter module 40 by surface micromachining would be to fabricate the first plate 44 at least in the P₂ layer 22 (possibly in the P₁ layer 18 as well, where the P₂ layer 22 is deposited directly on at least part of the P₁ layer 18). After at least the P₂ layer 22 has been patterned to define the perimeter of the first plate 44 and the various first flow ports 48 that extend through the first plate 44, the S₃ layer 24 may be deposited on top of the first plate 44 and into the first flow ports 48. Annular first troughs may then be patterned in the S₃ layer 24 to coincide with the location of the filtering walls 60, where these first troughs extend all the way down to the P₂ layer 22. Sacrificial material may be deposited in the bottom of these annular first troughs (the thickness of which will define the spacing between the ends of the filtering walls 60 and the first plate 44, or stated another way the height of the filter traps 64). The thickness of this deposition may be controlled with reasonable precision, or definable at small dimensions, to define a filter trap 64 of a desired height. One embodiment has the thickness of this deposition being no more than about 0.4 microns. Another embodiment has the thickness of this deposition being about 0.2 to about 0.3 microns. Yet another embodiment has the thickness of this deposition being about 0.1 microns or even less.

Annular second troughs may also be patterned in the above-noted S₃ layer 24 to coincide with the location of the annular structural interconnects 82, where these particular second troughs extend all the way down to the P₂ layer 22 as well. Similarly, apertures may be patterned in the S₃ layer 24 to coincide with the location of the structural interconnects 76, where these apertures also extend all the way down to the P₂ layer 22. The P₃ layer 26 may then be deposited on top of the S₃ layer 24 to define the second plate 52, as well as into the “partially filled” annular first troughs in the S₃ layer 24 (relating to the filtering walls 60), into the annular second troughs in the S₃ layer 24 (relating to the annular structural interconnects 82), and into the apertures in the S₃ layer 24 (relating to the structural interconnects 76). The deposition of structural material into the “partially filled” annular first troughs in the S₃ layer 24 is then what defines the filtering walls 60, the deposition of structural material into the annular second troughs in the S₃ layer 24 is then what defines the annular structural interconnects 82, and the deposition of structural material into the apertures is then what defines the structural interconnects 76. The second plate 52, the filtering walls 60, the annular structural interconnects 82, and the structural interconnects 76 may then be characterized as existing in a single fabrication level (P₃ layer 26 in the noted example), since they were all defined by a deposition of a structural material before having to form any overlying layer of a sacrificial material (e.g., from a single deposition of a structural layer or film). It should be noted that at least part of the S₃ layer 24 remains between the ends of the filtering walls 60 and the first plate 44 (prior to the release).

The first plate 44 and/or the second plate 52 of the MEMS filter modules 40 could also be fabricated in multiple structural layers or films at multiple fabrication levels as noted. For instance: the first plate 44 could be fabricated in both the P₂ layer 22 and P₁ layer 18, where the P₂ layer 22 is deposited directly on at least part of the P₁ layer 18 that is to define the first plate 44 (e.g., some material of the S₂ layer 20 could be encased at one or more locations between those portions of the P₂ layer 22 and the P₁ layer 18 that are to define the first plate 44, for any appropriate purpose); the first plate 44 could be fabricated in both the P₃ layer 26 and P₂ layer 22, where the P₃ layer 26 is deposited directly on at least part of the P₂ layer 22 that is to define the first plate 44 (e.g., some material of the S₃ layer 24 could be encased at one or more locations between those portions of the P₃ layer 26 and the P₂ layer 22 that are to define the first plate 44, for any appropriate purpose); and/or the second plate 52 could be fabricated in both the P₄ layer 30 and P₃ layer 26, where the P₄ layer 30 is deposited directly on at least part of the P₃ layer 26 that is to define the second plate 52 (e.g., some material of the S₄ layer 28 could be encased at one or more locations between those portions of the P₄ layer 30 and the P₃ layer 26 that are to define the second plate 52, for any appropriate purpose). Another option would be to form a particular component of the MEMS filter module 40 in multiple structural layers or films at different fabrication levels, but that are structurally interconnected in an appropriate manner (e.g., by one or more posts, columns or the like extending between). For instance: the first plate 44 could be formed in both the P₃ layer 26 and the P₂ layer 22 with one or more structural interconnections extending therebetween (that would pass through the S₃ layer 24); the second plate 52 could be formed in both the P₄ layer 30 and the P₃ layer 26 with one or more structural interconnections extending therebetween (that would pass through the S₄ layer 28). Generally, this can be done by forming appropriate cuts or openings down through the intermediate sacrificial layer (to expose the underlying structural layer and that will define such structural interconnections once the overlying structural layer is deposited both on top of the intermediate sacrificial layer and in the noted cuts or openings therein) before depositing the overlying structural layer. In any case, the first plate 44 and second plate 52 are fabricated at different fabrication levels, but are structurally interconnected by the annular structural interconnects 82 and the structural interconnects 76.

Notwithstanding the foregoing, the various components of the MEMS filter module 40 may be formed in different layers of a MEMS structure compared to what is been described herein. Furthermore, it will be appreciated that the various complements of the MEMS filter module 40 may be formed in a reverse order to that described herein.

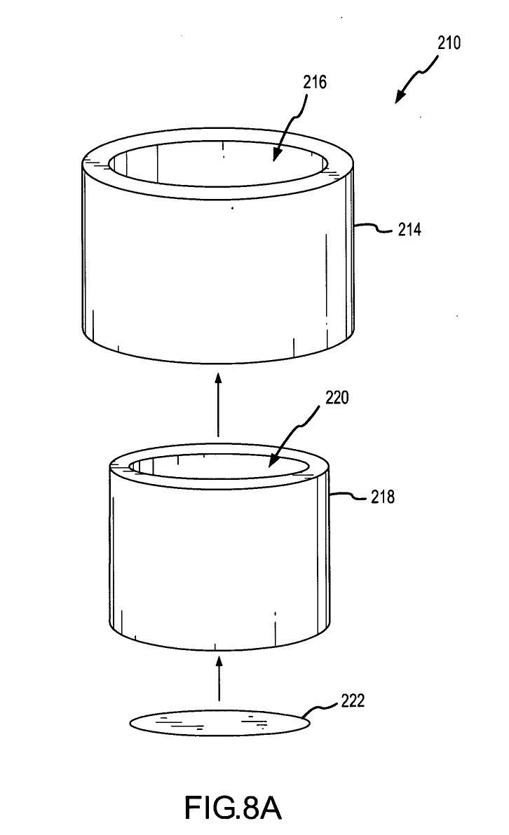

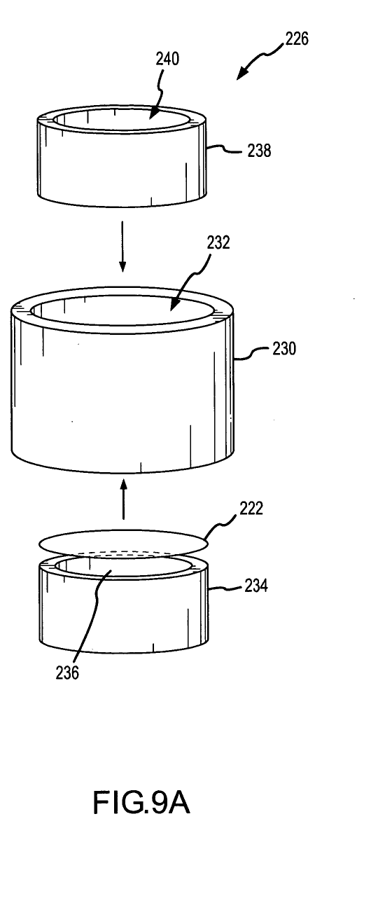

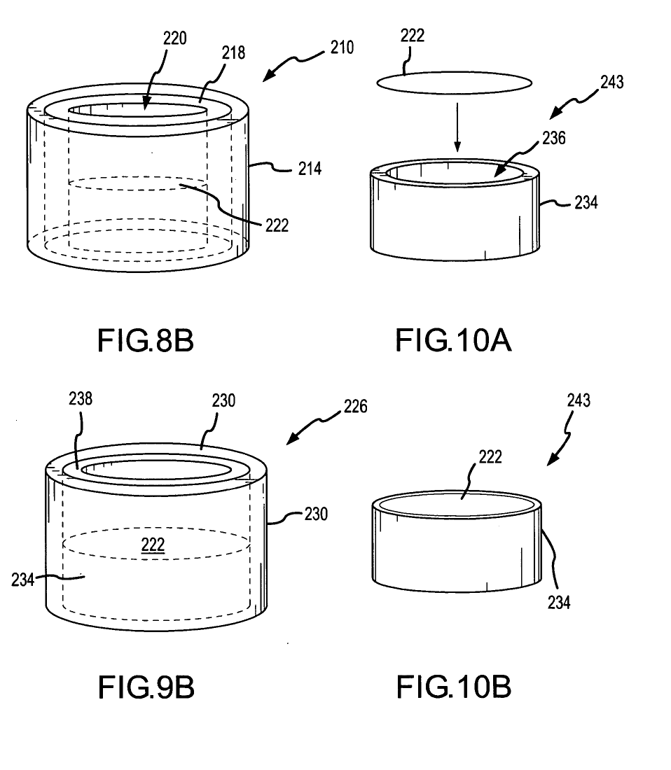

FIGS. 8A-B schematically represent one embodiment of a flow assembly 210 that may be used for any appropriate application (e.g., the flow assembly 210 may be disposed in a flow of any type, may be used to filter and/or control the flow of a fluid of any type, may be located in a conduit that fluidly interconnects multiple sources of any appropriate type (e.g., between multiple fluid or pressure sources (including where one is the environment), such as a man-made reservoir, a biological reservoir, the environment, or any other appropriate source), or any combination thereof). One example would be to dispose the flow assembly 210 in a conduit extending between the anterior chamber of an eye and a location that is exterior of the cornea of the eye. Another example would be to dispose the flow assembly 210 in a conduit extending between the anterior chamber of an eye and another location that is exterior of the sclera of the eye. Yet another example would be to dispose the flow assembly 210 in a conduit extending between the anterior chamber of an eye and another location within the eye (e.g., into Schlemm's canal) or body. In each of these examples, the conduit would provide an exit path for aqueous humor when installed for a glaucoma patient. That is, each of these examples may be viewed as a way of treating glaucoma or providing at least some degree of control of the intraocular pressure.

Components of the flow assembly 210 include an outer housing 214, an inner housing 218, and a MEMS flow module 222. The MEMS flow module 222 may be in the form of the MEMS filter module 40. The position of the MEMS flow module 222 and the inner housing 218 are at least generally depicted within the outer housing 214 in FIG. 8B to show the relative positioning of these components in the assembled condition—not to convey that the outer housing 214 needs to be in the form of a transparent structure. All details of the MEMS flow module 222 and the inner housing 218 are not necessarily illustrated in FIG. 8B.

The MEMS flow module 222 is only schematically represented in FIGS. 8A-B, and provides at least one of a filtering function and a pressure or flow regulation function. The MEMS flow module 222 may be of any appropriate design, size, shape, and configuration, and further may be formed from any material or combination of materials that are appropriate for use by the relevant microfabrication technology. Any appropriate coating or combination of coatings may be applied to exposed surfaces of the MEMS flow module 222 as well. For instance, a coating may be applied to improve the biocompatibility of the MEMS flow module 222, to make the exposed surfaces of the MEMS flow module 222 more hydrophilic, to reduce the potential for the MEMS flow module 222 causing any bio-fouling, or any combination thereof. In one embodiment, a self-assembled monolayer coating (e.g., poly-ethylene-glycol) is applied in any appropriate manner (e.g., liquid or vapor phase, with vapor phase being the preferred technique) to all exposed surfaces of the MEMS flow module 222. The main requirement of the MEMS flow module 222 is that it is a MEMS device.

The primary function of the outer housing 214 and inner housing 218 is to provide structural integrity for the MEMS flow module 222 or to support the MEMS flow module 222, and further to protect the MEMS flow module 222. In this regard, the outer housing 214 and inner housing 218 each will typically be in the form of a structure that is sufficiently rigid to protect the MEMS flow module 222 from being damaged by the forces that reasonably could be expected to be exerted on the flow assembly 210 during its assembly, as well as during use of the flow assembly 210 in the application for which it was designed.

The inner housing 218 includes a hollow interior or a flow path 220 that extends through the inner housing 218 (between its opposite ends in the illustrated embodiment). The MEMS flow module 222 may be disposed within the flow path 220 through the inner housing 218 in any appropriate manner and at any appropriate location within the inner housing 218 (e.g., at any location so that the inner housing 218 is disposed about the MEMS flow module 222). Preferably, the MEMS flow module 222 is maintained in a fixed position relative to the inner housing 218. For instance, the MEMS flow module 222 may be attached or bonded to an inner sidewall or a flange formed on this inner sidewall of the inner housing 218, a press-fit could be provided between the inner housing 218 and the MEMS flow module 222, or a combination thereof. The MEMS flow module 222 also could be attached to an end of the inner housing 218 in the manner of the embodiment of FIGS. 10A-B that will be discussed in more detail below.

The inner housing 218 is at least partially disposed within the outer housing 214 (thereby encompassing having the outer housing 214 being disposed about the inner housing 218 along the entire length of the inner housing 218, or only along a portion of the length of the inner housing 218). In this regard, the outer housing 214 includes a hollow interior 216 for receiving the inner housing 218, and possibly to provide other appropriate functionality (e.g., a flow path fluidly connected with the flow path 220 through the inner housing 218). The outer and inner sidewalls of the outer housing 214 may be cylindrical or of any other appropriate shape, as may be the outer and inner sidewalls of the inner housing 218. The inner housing 218 may be retained relative to the outer housing 214 in any appropriate manner. For instance, the inner housing 218 may be attached or bonded to an inner sidewall of the outer housing 214, a press-fit could be provided between the inner housing 218 and the outer housing 214, a shrink fit could be provided between the outer housing 214 and the inner housing 218, or a combination thereof.

The inner housing 218 is likewise only schematically represented in FIGS. 8A-B, and it may be of any appropriate shape/configuration, of any appropriate size, and formed from any material or combination of materials (e.g., polymethylmethacrylate (PMMA), ceramics, silicon, titanium, and other implantable metals and plastics). Typically its outer contour will be adapted to match the inner contour of the outer housing 214 in which it is at least partially disposed. In one embodiment, the illustrated cylindrical configuration for the inner housing 218 is achieved by cutting an appropriate length from hypodermic needle stock. The inner housing 218 also may be microfabricated into the desired/required shape (e.g., using at least part of a LIGA process). However, any way of making the inner housing 218 may be utilized. It should also be appreciated that the inner housing 218 may include one or more coatings as desired/required as well (e.g., an electroplated metal; a coating to improve the biocompatibility of the inner housing 218, to make the exposed surfaces of the inner housing 218 more hydrophilic, to reduce the potential for the inner housing 218 causing any bio-fouling, or any combination thereof). In one embodiment, a self-assembled monolayer coating (e.g., poly-ethylene-glycol) is applied in any appropriate manner (e.g., liquid or vapor phase, with vapor phase being the preferred technique) to all exposed surfaces of the inner housing 218.

The outer housing 214 likewise is only schematically represented in FIGS. 8A-B, and it may be of any appropriate shape/configuration, of any appropriate size, and formed from any material or combination of materials (e.g., polymethylmethacrylate (PMMA), ceramics, silicon, titanium, and other implantable metals and plastics). Typically its outer contour will be adapted to match the inner contour of the housing or conduit in which it is at least partially disposed or otherwise mounted. The outer housing 214 also may be microfabricated into the desired/required shape (e.g., using at least part of a LIGA process). However, any way of making the outer housing 214 may be utilized. It should also be appreciated that the outer housing 214 may include one or more coatings as desired/required as well (e.g., an electroplated metal; a coating to improve the biocompatibility of the outer housing 214, to make the exposed surfaces of the outer housing 214 more hydrophilic, to reduce the potential for the outer housing 214 causing any bio-fouling, or any combination thereof). In one embodiment, a self-assembled monolayer coating (e.g., poly-ethylene-glycol) is applied in any appropriate manner (e.g., liquid or vapor phase, with vapor phase being the preferred technique) to all exposed surfaces of the outer housing 214.

Another embodiment of a flow assembly is illustrated in FIGS. 9A-B (only schematic representations), and is identified by reference numeral 226. The flow assembly 226 may be used for any appropriate application (e.g., the flow assembly 226 may be disposed in a flow of any type, may be used to filter and/or control the flow of a fluid of any type, may be located in a conduit that fluidly interconnects multiple sources of any appropriate type (e.g., multiple fluid or pressure sources (including where one is the environment), such as a man-made reservoir, a biological reservoir, the environment, or any other appropriate source), or any combination thereof). The above-noted applications for the flow assembly 210 are equally applicable to the flow assembly to 226. The types of coatings discussed above in relation to the flow assembly 210 may be used by the flow assembly 226 as well.

Components of the flow assembly 226 include an outer housing 230, a first inner housing 234, a second inner housing 238, and the MEMS flow module 222. The MEMS flow 222 and the inner housings 234, 238 are at least generally depicted within the outer housing 230 in FIG. 9B to show the relative positioning of these components in the assembled condition—not to convey that the outer housing 230 needs to be in the form of a transparent structure. All details of the MEMS flow module 222 and the inner housings 234, 238 are not necessarily illustrated in FIG. 9B.

The primary function of the outer housing 230, first inner housing 234, and second inner housing 238 is to provide structural integrity for the MEMS flow module 222 or to support the MEMS flow module 222, and further to protect the MEMS flow module 222. In this regard, the outer housing 230, first inner housing 234, and second inner housing 238 each will typically be in the form of a structure that is sufficiently rigid to protect the MEMS flow module 222 from being damaged by the forces that reasonably could be expected to be exerted on the flow assembly 226 during its assembly, as well as during use of the flow assembly 226 in the application for which it was designed.

The first inner housing 234 includes a hollow interior or a flow path 236 that extends through the first inner housing 234. Similarly, the second inner housing 238 includes a hollow interior or a flow path 240 that extends through the second inner housing 238. The first inner housing 234 and the second inner housing 238 are disposed in end-to-end relation, with the MEMS flow module 222 being disposed between adjacent ends of the first inner housing 234 and the second inner housing 238. As such, a flow progressing through the first flow path 236 to the second flow path 240, or vice versa, passes through the MEMS flow module 222.

Preferably, the MEMS flow module 222 is maintained in a fixed position relative to each inner housing 234, 238, and its perimeter does not protrude beyond the adjacent sidewalls of the inner housings 234, 238 in the assembled and joined condition. For instance, the MEMS flow module 222 may be bonded to at least one of, but more preferably both of, the first inner housing 234 (more specifically one end thereof) and the second inner housing 238 (more specifically one end thereof) to provide structural integrity for the MEMS flow module 222 (e.g., using cyanoacrylic esters, thermal bonding, UV-curable epoxies, or other epoxies). Another option would be to fix the position the MEMS flow module 222 in the flow assembly 226 at least primarily by fixing the position of each of the inner housings 234, 238 relative to the outer housing 230 (i.e., the MEMS flow module 222 need not necessarily be bonded to either of the housings 234, 238). In one embodiment, an elastomeric material may be disposed between the MEMS flow module 222 and the first inner housing 234 to allow the first inner housing 234 with the MEMS flow module 222 disposed thereon to be pushed into the outer housing 230 (e.g., the elastomeric material is sufficiently “tacky” to at least temporarily retain the MEMS flow module 222 in position relative to the first inner housing 234 while being installed in the outer housing 230). The second inner housing 238 also may be pushed into the outer housing 230 (before, but more likely after, the first inner housing 234 is disposed in the outer housing 230) to “sandwich” the MEMS flow module 222 between the inner housings 234, 238 at a location that is within the outer housing 230 (i.e., such that the outer housing 230 is disposed about MEMS flow module 222). The MEMS flow module 222 would typically be contacted by both the first inner housing 234 and the second inner housing 238 when disposed within the outer housing 230. Fixing the position of each of the first inner housing 234 and the second inner housing 238 relative to the outer housing 230 will thereby in effect fix the position of the MEMS flow module 222 relative to the outer housing 230. Both the first inner housing 234 and second inner housing 238 are at least partially disposed within the outer housing 230 (thereby encompassing the outer housing 230 being disposed about either or both housings 234, 238 along the entire length thereof, or only along a portion of the length of thereof), again with the MEMS flow module 222 being located between the adjacent ends of the first inner housing 234 and the second inner housing 238. In this regard, the outer housing 230 includes a hollow interior 232 for receiving at least part of the first inner housing 234, at least part of the second inner housing 238, and the MEMS flow module 222 disposed therebetween, and possibly to provide other appropriate functionality (e.g., a flow path fluidly connected with the flow paths 236, 240 through the first and second inner housings 234, 238, respectively). The outer and inner sidewalls of the outer housing 230 may be cylindrical or of any other appropriate shape, as may be the outer and inner sidewalls of the inner housings 234, 238. Both the first inner housing 234 and the second inner housing 238 may be secured to the outer housing 230 in any appropriate manner, including in the manner discussed above in relation to the inner housing 218 and the outer housing 214 of the embodiment of FIGS. 8A-B.

Each inner housing 234, 238 is likewise only schematically represented in FIGS. 9A-B, and each may be of any appropriate shape/configuration, of any appropriate size, and formed from any material or combination of materials in the same manner as the inner housing 218 of the embodiment of FIGS. 7-8. Typically the outer contour of both housings 234, 238 will be adapted to match the inner contour of the outer housing 230 in which they are at least partially disposed. In one embodiment, the illustrated cylindrical configuration for the inner housings 234, 238 is achieved by cutting an appropriate length from hypodermic needle stock. The inner housings 234, 238 each also may be microfabricated into the desired/required shape (e.g., using at least part of a LIGA process). However, any way of making the inner housings 234, 238 may be utilized. It should also be appreciated that the inner housings 234, 238 may include one or more coatings as desired/required as well in accordance with the foregoing.

The outer housing 230 is likewise only schematically represented in FIGS. 9A-B, and it may be of any appropriate shape/configuration, of any appropriate size, and formed from any material or combination of materials in the same manner as the outer housing 214 of the embodiment of FIGS. 8A-B. Typically the outer contour of the outer housing 230 will be adapted to match the inner contour of the housing or conduit in which it is at least partially disposed or otherwise mounted. The outer housing 230 may be microfabricated into the desired/required shape (e.g., using at least part of a LIGA process). However, any way of making the outer housing 230 may be utilized. It should also be appreciated that the outer housing 230 may include one or more coatings as desired/required in accordance with the foregoing.

Another embodiment of a flow assembly is illustrated in FIGS. 10A-B (only schematic representations), and is identified by reference numeral 243. The flow assembly 243 may be used for any appropriate application (e.g., the flow assembly 243 may be disposed in a flow of any type, may be used to filter and/or control the flow of a fluid of any type, may be located in a conduit that fluidly interconnects multiple sources of any appropriate type (e.g., between multiple fluid or pressure sources, such as a man-made reservoir, a biological reservoir, the environment, or any other appropriate source), or any combination thereof). Components of the flow assembly 243 include the above-noted housing 234 and the MEMS flow module 222 from the embodiment of FIGS. 9A-B. In the case of the flow assembly 243, the MEMS flow module 222 is attached or bonded to one end of the housing 234 (e.g., using cyanoacrylic esters, thermal bonding, UV-curable epoxies, or other epoxies). The flow assembly 243 may be disposed within an outer housing in the manner of the embodiments of FIGS. 8A-9B, or could be used “as is.” The above-noted applications for the flow assembly 210 are equally applicable to the flow assembly 243. The types of coatings discussed above in relation to the flow assembly 210 may be used by the flow assembly 243 as well.

One particularly desirable application for the flow assemblies 210, 226, and 243 of FIGS. 8A-10B, as discussed above, is to regulate pressure within the anterior chamber of an eye. That is, they may be disposed in an exit path through which aqueous humor travels to treat a glaucoma patient. Preferably, the flow assemblies 210, 226, 243 each provide a bacterial filtration function to reduce the potential for developing an infection within the eye. Although the various housings and MEMS filter modules used by the flow assemblies 210, 226, and 243 each may be of any appropriate color, it may be desirable for the color to be selected so as to “blend in” with the eye to at least some extent.

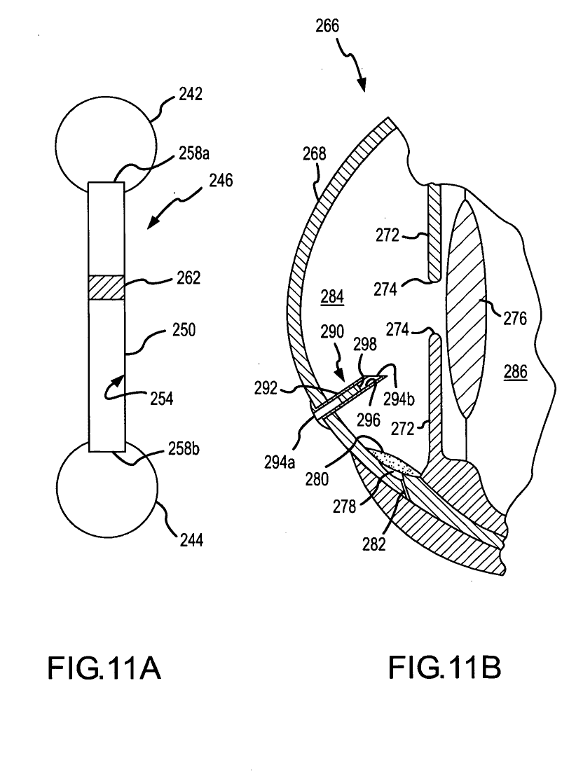

An example of the above-noted application is schematically illustrated in FIG. 11A. Here, an anterior chamber 242 of a patient's eye (or other body region for that matter—a first body region) is fluidly interconnected with an appropriate drainage area 244 by an implant 246 (a “glaucoma implant” for the specifically noted case). The drainage area 244 may be any appropriate location, such as externally of the eye (e.g., on an exterior surface of the cornea), within the eye (e.g., Schlemm's canal), or within the patient's body in general (a second body region).

Generally, the implant 246 includes a conduit 250 having a pair of ends 258a, 258b, with a flow path 254 extending therebetween. The size, shape, and configuration of the conduit 250 may be adapted as desired/required, including to accommodate the specific drainage area 244 being used. Representative configurations for the conduit 250 are disclosed in U.S. Patent Application Publication No. 2003/0212383, as well as U.S. Pate. Nos. 3,788,327; 5,743,868; 5,807,302; 6,626,858; 6,638,239; 6,533,768; 6,595,945; 6,666,841; and 6,736,791, the entire disclosures of which are incorporated by reference in their entirety herein.

A flow assembly 262 is disposed within the flow path 254 of the conduit 250. All flow leaving the anterior chamber 242 through the implant 246 is thereby directed through the flow assembly 262. Similarly, any flow from the drainage area 244 into the implant 246 will have to pass through the flow assembly 262. The flow assembly 262 may be retained within the conduit 250 in any appropriate manner and at any appropriate location (e.g., it could be disposed on either end 258a, 258b, or any intermediate location therebetween). The flow assembly 262 may be in the form of any of the flow assemblies 210, 226, or 243 discussed above, replacing the MEMS flow module 222 with the MEMS filter module 40. Alternatively, the flow assembly 262 could simply be in the form of the MEMS filter module 40. Any appropriate coating may be applied to at least those surfaces of the implant 246 that would be exposed to biological material/fluids, including without limitation a coating that improves biocompatibility, that makes such surfaces more hydrophilic, and/or that reduces the potential for bio-fouling. In one embodiment, a self-assembled monolayer coating (e.g., poly-ethylene-glycol) is applied in any appropriate manner (e.g., liquid or vapor phase, with vapor phase being the preferred technique) to the noted surfaces.

FIG. 11B illustrates a representative embodiment in accordance with FIG. 11A. Various portions of the eye 266 are identified in FIG. 11B, including the cornea 268, iris 272, pupil 274, lens 276, anterior chamber 284, vitreous body 286, Schlemm's canal 278, trabecular meshwork 280, and aqueous veins 282. Here, an implant or shunt 290 having an appropriately-shaped conduit 292 is directed through the cornea 268. The conduit 292 may be in any appropriate form, but will typically include at least a pair of ends 294a, 294b, as well as a flow path 296 extending therebetween. End 294a is disposed on the exterior surface of the cornea 268, while end 294b is disposed within the anterior chamber 284 of the eye 266.

A flow assembly 298 is disposed within the flow path 296 of the conduit 292. All flow leaving the anterior chamber 284 through the shunt 290 is thereby directed through the flow assembly 298. Similarly, any flow from the environment back into the shunt 290 will have to pass through the flow assembly 298 as well. Preferably, the flow assembly 298 provides a bacterial filtration function to reduce the potential for developing an infection within the eye when using the implant 290. The flow assembly 298 may be retained within the conduit 292 in any appropriate manner and at any appropriate location (e.g., it could be disposed on either end 294a, 294b, or any an intermediate location therebetween). The flow assembly 298 may be in the form of any of the flow assemblies 210, 226, or 243 discussed above, replacing the MEMS flow module 222 with the MEMS filter module 40. Alternatively, the flow assembly 298 could simply be in the form of the MEMS filter module 40. Any appropriate coating may be applied to at least those surfaces of the shunt 290 that would be exposed to biological material/fluids, including without limitation a coating that improves biocompatibility, that makes such surfaces more hydrophilic, and/or that reduces the potential for bio-fouling. In one embodiment, a self-assembled monolayer coating (e.g., poly-ethylene-glycol) is applied in any appropriate manner (e.g., liquid or vapor phase, with vapor phase being the preferred technique) to the noted surfaces.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

What is claimed is:

1. A MEMS filter module, comprising:
a first plate comprising a plurality of first flow ports;
a second plate comprising a plurality of second flow ports;
a plurality of filtering walls, wherein each said filtering wall extends along at least part of one of a plurality of at least generally concentrically disposed filtering wall reference circles, wherein each of said plurality of filtering wall reference circles has at least one associated said filtering wall, wherein each said filtering wall extends from said second plate in a direction of said first plate, and wherein a space between said first plate and each said filtering wall defines a filter trap.

2. The MEMS filter module of claim 1, wherein each said first flow port is disposed on one of a plurality of at least generally concentrically disposed first reference circles, wherein each said first reference circle has at least one associated said first flow port, wherein each said first reference circle is laterally offset from each said filtering wall reference circle such that each said first flow port is laterally offset from each said filtering wall, and wherein a lateral dimension is perpendicular to a thickness dimension of said first and second plates.

3. The MEMS filter module of claim 2, wherein each said second flow port is disposed on one of a plurality of at least generally concentrically disposed second reference circles, wherein each said second reference circle has at least one associated said second flow port, wherein each said second reference circle is laterally offset from each said first reference circle and is laterally offset from each said filtering wall reference circle such that each said second flow port is laterally offset from each said filtering wall and is offset from each said first flow port.

4. The MEMS filter module of claim 1, wherein each said second flow port is disposed on one of a plurality of at least generally concentrically disposed second reference circles, wherein each said second reference circle has at least one associated said second flow port, wherein each said second reference circle is laterally offset from each said filtering wall reference circle such that each said second flow port is laterally offset from each said filtering wall, and wherein a lateral dimension is perpendicular to a thickness dimension of said first and second plates.

5. The MEMS filter module of claim 1, wherein a space between each adjacent pair of said filtering wall reference circles comprises a chamber, wherein a first member of each adjacent pair of said chambers is associated with only a first flow port type and a second member of each said adjacent pair of said chambers is associated with only a second flow port type, wherein said plurality of first flow ports are of said first flow port type, and wherein said plurality of second flow ports are of said second flow port type.

6. The MEMS filter module of claim 5, wherein each said chamber is associated with either a plurality of said first flow ports or a plurality of said second flow ports.

7. The MEMS filter module of claim 5, wherein each said chamber is associated with at least one said first flow port or at least one said second flow port.

8. The MEMS filter module of claim 1, further comprising a plurality of first flow port chambers and a plurality of second flow port chambers, wherein each said first flow port chamber and each said second flow port chamber is bounded in one dimension by said first and second plates and is bounded in a second dimension by each said filtering wall associated with adjacent pairs of said filtering wall reference circles, wherein said first and second flow port chambers are disposed in alternating relation, wherein each said first flow port chamber is associated with only a first flow port type, wherein each said second flow port chamber is associated with only a second flow port type, wherein said plurality of first flow ports are of said first flow port type, and wherein said plurality of second flow ports are of said second flow port type.

9. The MEMS filter module of claim 1, wherein each said filtering wall is annular, such that said plurality of filtering walls are at least generally concentrically disposed.

10. The MEMS filter module claim 9, wherein said plurality of filtering walls are equally spaced.

11. The MEMS filter module of claim 1, wherein a plurality of said filtering walls are disposed on a common said filtering wall reference circle and spaced from each other.

12. The MEMS filter module of claim 1, wherein each said filtering wall terminates prior to reaching said first plate.

13. The MEMS filter module of claim 1, further comprising first and second fabrication levels, wherein said first fabrication level comprises said first plate and said plurality of first flow ports, and wherein said second fabrication level comprises said second plate, said plurality of second flow ports, and said plurality of filtering walls.

14. The MEMS filter module of claim 1, further comprising a plurality of structural interconnects extending between said first and second plates.

15. The MEMS filter module of claim 1, further comprising at least one annular structural interconnect extending between said first and second plates, wherein each of said plurality of first flow ports, said plurality of second flow ports, and said plurality of filtering walls are disposed inwardly of said at least one annular structural interconnect.

16. An implant associated with a first body region and that comprises said MEMS filter module of claim 1 and a conduit, wherein said conduit comprises a flow path that is adapted to fluidly interconnect with the first body region, and wherein said MEMS filter module is disposed in said flow path.

17. The implant of claim 16, further comprising at least one housing, wherein said at least one housing is disposed within said conduit, and wherein said MEMS filter module interfaces with said at least one housing.

18. An implant installable in a human eye and comprising said MEMS filter module of claim 1 and a conduit, wherein said conduit comprises a flow path that is adapted to fluidly interconnect with an anterior chamber of the human eye when said implant is installed, and wherein said MEMS filter module is disposed in said flow path.

19. A MEMS filter module, comprising:
a first plate comprising a plurality of first flow ports;
a second plate comprising a plurality of second flow ports;
a plurality of annular filtering walls that are disposed about a common point, wherein each said filtering wall extends from said second plate in a direction of said first plate, and wherein a space between said first plate and each said filtering wall defines a filter trap.

20. The MEMS filter module of claim 19, wherein a space between each adjacent pair of said filtering walls comprises a chamber, wherein a first member of each adjacent pair of said chambers is associated with only a first flow port type and a second member of each said adjacent pair of said chambers is associated with only a second flow port type, wherein said plurality of first flow ports are of said first flow port type, and wherein said plurality of second flow ports are of said second flow port type.

21. The MEMS filter module of claim 20, wherein each said chamber is associated with either a plurality of said first flow ports or a plurality of said second flow ports.

22. The MEMS filter module of claim 20, wherein each said chamber is associated with at least one said first flow port or at least one said second flow port.

23. The MEMS filter module of claim 19, further comprising a plurality of first flow port chambers and a plurality of second flow port chambers, wherein each said first flow port chamber and each said second flow port chamber is bounded in one dimension by said first and second plates and is bounded in a second dimension by an adjacent pair of said filtering walls, wherein said first and second flow port chambers are disposed in alternating relation, wherein each said first flow port chamber is associated with only a first flow port type, wherein each said second flow port chamber is associated with only a second flow port type, wherein said plurality of first flow ports are of said first flow port type, and wherein said plurality of second flow ports are of said second flow port type.

24. The MEMS filter module of claim 19, wherein said plurality of filtering walls are at least generally concentrically disposed.

25. The MEMS filter module claim 24, wherein said plurality of filtering walls are equally spaced.

26. The MEMS filter module of claim 19, wherein each said filtering wall terminates prior to reaching said first plate.

27. The MEMS filter module of claim 19, further comprising first and second fabrication levels, wherein said first fabrication level comprises said first plate and said plurality of first flow ports, and wherein said second fabrication level comprises said second plate, said plurality of second flow ports, and said plurality of filtering walls.

28. The MEMS filter module of claim 19, further comprising a plurality of structural interconnects extending between said first and second plates.

29. The MEMS filter module of claim 19, further comprising at least one annular structural interconnect extending between said first and second plates, wherein each of said plurality of first flow ports, said plurality of second flow ports, and said plurality of filtering walls are disposed inwardly of said at least one annular structural interconnect.

30. An implant associated with a first body region and that comprises said MEMS filter module of claim 19 and a conduit, wherein said conduit comprises a flow path that is adapted to fluidly interconnect with the first body region, and wherein said MEMS filter module is disposed in said flow path.

31. The implant of claim 30, further comprising at least one housing, wherein said at least one housing is disposed within said conduit, and wherein said MEMS filter module interfaces with said at least one housing.

32. An implant installable in a human eye and comprising said MEMS filter module of claim 19 and a conduit, wherein said conduit comprises a flow path that is adapted to fluidly interconnect with an anterior chamber of the human eye when said implant is installed, and wherein said MEMS filter module is disposed in said flow path.
